(19) United States
(12) Patent Application Publication
(10) Pub. No.: US 2025/0295178 A1
(43) Pub. Date: Sep. 25, 2025

(54) SMOKING CESSATION SYSTEM

(71) Applicant: Xerbal USA, LLC., Fullerton, CA (US)

(72) Inventors: Peter Matravers, Fullerton, CA (US); Joe Wong, Fullerton, CA (US); Cary Tu, Hong Kong (HK)

(21) Appl. No.: 19/087,279

(22) Filed: Mar. 21, 2025

Related U.S. Application Data
(60) Provisional application No. 63/568,965, filed on Mar. 22, 2024.
(60) Provisional application No. 63/747,805, filed on Jan. 21, 2025.

(51) Int. Cl. A24F 47/00; A24D 3/14; G16H 20/10

(52) U.S. Cl. CPC A24F 47/00; A24D 3/14; G16H 20/10

(57) ABSTRACT

This application relates to novel processes for treating persons who are cigarette smokers or are otherwise suffering from nicotine addiction. This application also relates to novel computer programs for artificial intelligence for the treatment of nicotine addition, as well as novel data structures and methods for the implementation of same. This application also relates to a novel formulation of liquid drops for application to filter cigarettes to block nicotine and/or tar from user inhalation.

### CROSS-REFERENCE TO RELATED APPLICATIONS

This application claims priority to U.S. Provisional Patent Application No. 63/568,965; filed on Mar. 22, 2024; and U.S. Provisional Patent Application No. 63/747,805; filed on Jan. 21, 2025; both of which are incorporated by reference herein in their entirety.

### FIELD OF THE DISCLOSURE

This application relates to novel processes for treating persons who are cigarette smokers or are otherwise suffering from nicotine addiction. This application also relates to novel computer programs for artificial intelligence for the treatment of nicotine addition, as well as novel data structures and methods for the implementation of same. This application also relates to a novel formulation of liquid drops for application to filter cigarettes to block nicotine and/or tar from user inhalation.

### BACKGROUND

It is estimated that there are 1.3 billion cigarette smokers in the world. Most cigarette smokers admit that they want to quit and have made attempts to quit smoking. Indeed, some estimates suggest that about 68% of cigarette smokers, i.e., almost 800 million people, want to quit smoking. In general, their success rate is extremely low, around 4-7%.

One issue is that only a small percentage of smokers have access to quality quit-smoking products. It appears that many smokers are still trying to quit unassisted, rather than utilizing smoking cessation aids or other forms of assistance. The lack of access is due to many different factors, including the expense of the currently marketed smoking cessation aids, the lack of widespread distribution of smoking cessation aides and regulatory obstacles to drug-based aids.

Another issue is that many existing quit-smoking products, like e-cigarettes and vapes, and quit-smoking therapies on the market are often of low-quality, made with petrochemical and artificial flavors, and do not work in support of consumers' health. Nicotine supplement therapies, gums and patches often do not work long term, because they treat only the physical craving for nicotine, but do not adequately address social and emotion factors. Thus, relapses back to cigarette smoking are very common.

Yet another issue is the need for specific computer algorithms, artificial intelligence programs, and data structures that are focused on addressing the wholistic needs of a cigarette smoker who is attempting to quit smoking. To the best of Applicant's knowledge, there are not currently any specific computer algorithms, artificial intelligence programs, or data structures that can gather information, store information, and process that information relating to a smoker's physical health, mental health, and spiritual health, and then be able to provide support and treatment for all of these aspects of a patient during their attempt to quit smoking.

Accordingly, there is a need for an improved method of smoking cessation, and tools and products for smoking cessation, that address the issues and disadvantages of prior art approaches discussed above.

### SUMMARY

Embodiments disclosed herein address the needs described above and relate to an improved method, algorithm, computer program, artificial intelligence, data structures, and system for assisting cigarette smokers to quit smoking. Embodiments disclosed herein also relate to a novel formulation of liquid drops for application to filter cigarettes to block nicotine and/or tar from user inhalation.

### BRIEF DESCRIPTION OF THE DRAWINGS

In order that the advantages of the embodiments disclosed herein will be readily understood, a more particular description of these embodiments will be rendered by reference to specific embodiments illustrated in the appended drawings. Understanding that these drawings depict only typical embodiments and are not therefore to be considered limiting of its scope, these embodiments will be described and explained with additional specificity and detail through use of the accompanying drawings, in which:

FIG. 1 illustrates an innovative Behavior Analytics AI (BAAI) driven smoking cessation model that utilizes behavior analytics algorithms derived from the successes and failures of smokers attempting to quit;

FIG. 2 depicts a schematic diagram of a system designed to gather infused data from various sources, including mobile phones, wearable devices, person-to-person interactions, computers, and more;

FIG. 3 illustrates the Xerbal data structure which refers to the representation and key features of the data collected and used to analyze and predict smoking behavior patterns; and

FIG. 4 shows an overview of the Behavior Analytics & AI Applications in the Xerbal Model.

### DETAILED DESCRIPTION OF THE EMBODIMENTS

The description that follows is presented to enable one skilled in the art to make and use the disclosed embodiments, and is provided in the context of a particular application and its requirements. Various modifications to the disclosed embodiments will be apparent to those skilled in the art, and the general principles discussed may be applied to other embodiments and applications without departing from the scope and spirit of the invention. Therefore, the invention is not intended to be limited to the embodiments disclosed, but the invention is to be given the largest possible scope which is consistent with the principles and features described herein.

Embodiments disclosed herein relate to a method of treating smokers for cigarettes and nicotine addiction, for providing or facilitating the effects of smoking cessation via a system of interventions.

Embodiments disclosed herein also include the related natural Vape device for Vape users or Nicotine/Tar block drops for cigarette smokers, a cutting-edge AI program for product selection and maintenance support, as well as a scientifically validated questionnaire to discover smoker's mind-set and degree of dependence as part of the AI program.

This system makes it possible for a smoker to gradually reduce amounts of nicotine over time thereby allowing the smoker to be gradually weaned off dependence on nicotine and quit smoking naturally.

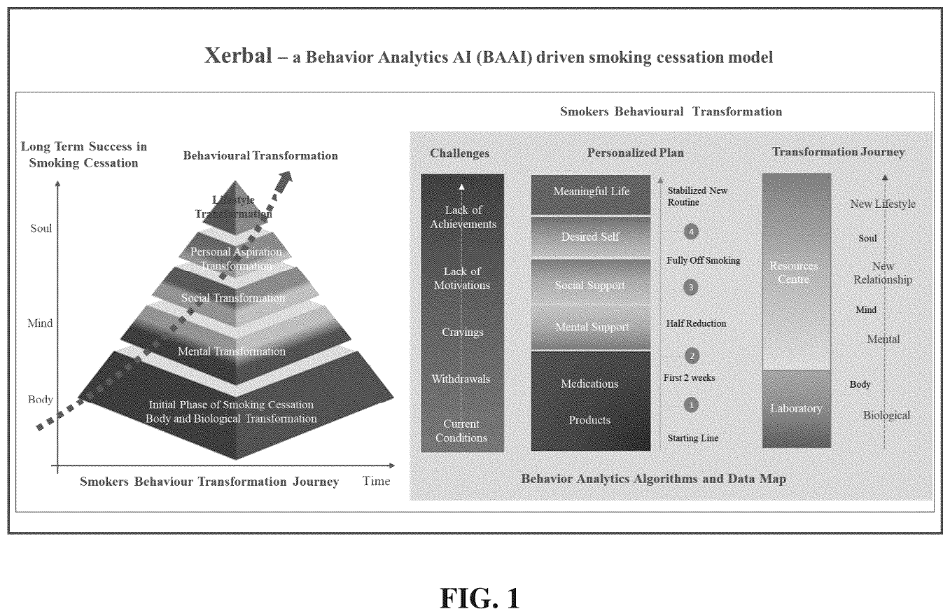

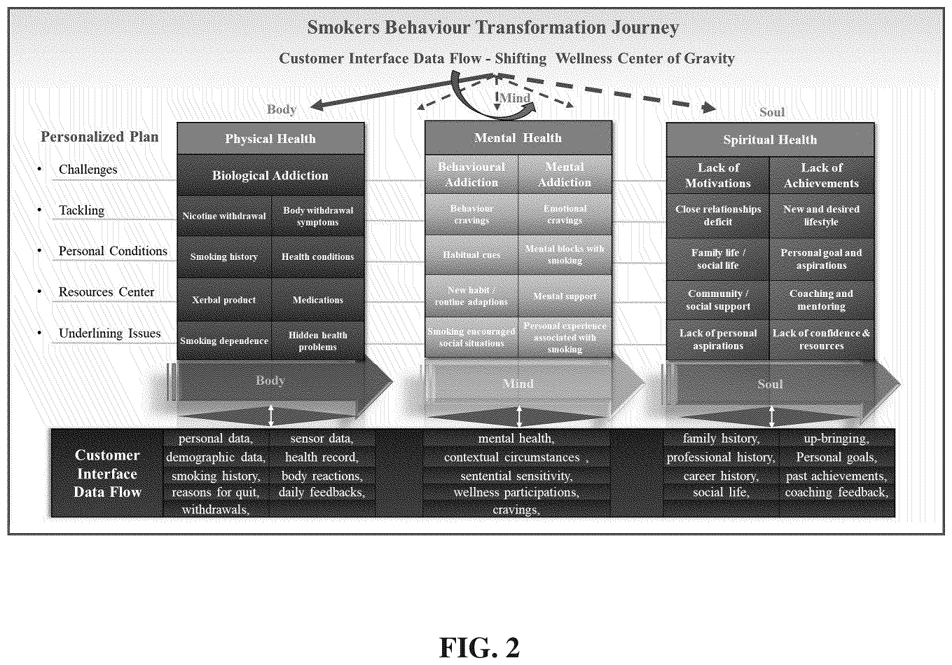

With reference to FIG. 1, the Xerbal model captures patterns and correlations in the behavior of individuals throughout their personal transformation journeys, addressing physical, emotional, and behavioral addiction. This journey aims to shift the Wellness Center of Gravity (see FIG. 2) from body to mind to soul, encompassing the holistic experiences of smokers.

The uniqueness of each smoker's transformation journey is emphasized, considering factors such as physical condition, smoking history, health status, withdrawal symptoms, cravings, motivations, support systems, contextual influences, cultural and religious beliefs, and personal values. These behavioral variances further differentiate the individualized transformation journey for each smoker.

To address this individuality, a personalized cessation program for each smoker is ideal. The implementation of a Behavior Analytics AI (BAAI) driven smoking cessation model enables the practical delivery of tailored solutions on a massive scale. This breakthrough allows personalized programs to be delivered simultaneously to an unlimited number of smokers, eliminating the conventional dilemma of choosing between personalization on a small scale or mass production through standardization.

The embodiments disclosed herein explain the value of using behavior analytics algorithms in an AI-driven smoking cessation program, providing personalized solutions, and addressing the diverse needs of smokers on a large scale and improving upon the low success rate of smoking cessation solutions currently available in the field.

With reference to FIG. 2, FIG. 2 depicts a schematic diagram of a system designed to gather infused data from various sources, including mobile phones, wearable devices, person-to-person interactions, computers, and more. These interfaces allow smokers to interact with the system, enabling data collection through questionnaires, chat messages, interviews, behavioral responses, device sensors, social media, visual records, medical and health records, and other means. This data serves as a crucial input for the behavioral analytics algorithms, enabling the creation of personalized cessation programs for each unique smoker.

The data flow within the system plays a vital role in driving the behavioral analytics algorithms, which analyze the collected data to generate customized programs. As the cessation program progresses, different sets of data are gathered at various stages. This dynamic data collection aids in shifting the smokers' Wellness Center of Gravity, progressively transitioning their focus from physical health to mental and ultimately spiritual well-being.

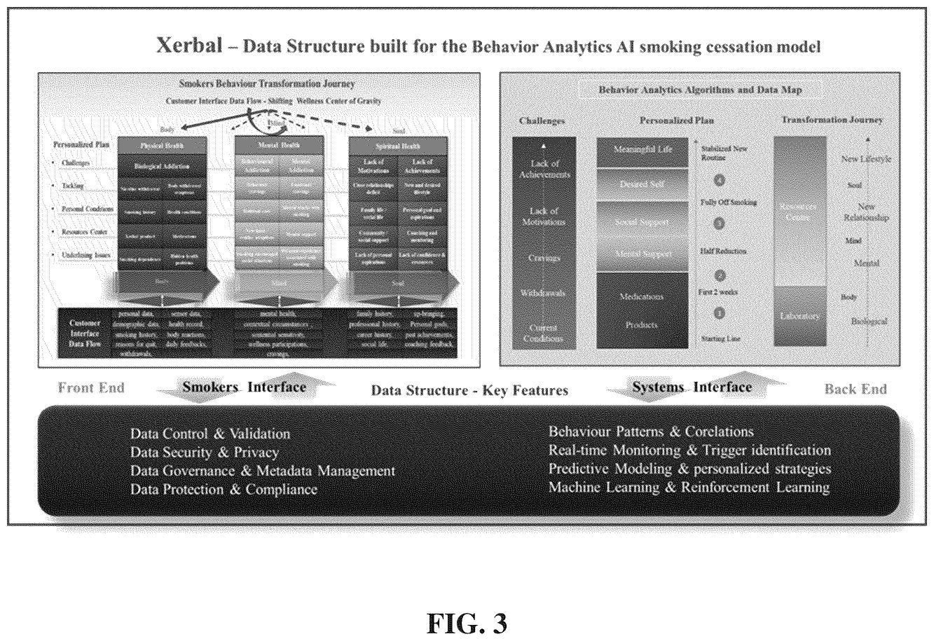

FIG. 3 illustrates the Xerbal data structure which refers to the representation and key features of the data collected and used to analyze and predict smoking behavior patterns. It serves as a framework that organizes and defines the relationships between different data elements, variables, and features relevant to smoking cessation. The Xerbal model presents a novel system and method for comprehensive data collection, management, encompassing data control and validation, data security and privacy, data governance and metadata management, as well as data protection and compliance. The database and structure aim to address the challenges associated with ensuring the integrity, security, and regulatory compliance of data in various domains. By integrating these four key aspects into a unified framework, our system provides a robust and efficient solution to effectively manage our data assets and mitigate risks.

The FIG. 3 Xerbal model presents a groundbreaking behavior analytics AI system that leverages:
Behavior Patterns & Correlations:
   Our data structure supports the advanced algorithms to analyze behavior patterns and identify correlations within vast datasets. By processing and analyzing large volumes of structured and unstructured data, including user interactions, and social media activity, the system uncovers hidden patterns and identifies meaningful correlations. These insights provide valuable information for understanding smoker behaviors along the transformation journey.
Real-time Monitoring & Trigger Identification:
   The data system continuously monitors real-time data streams, capturing and analyzing behavioral events as they occur. Through advanced data processing and pattern recognition by the Behavior Analytics algorithms, our system identifies triggers and anomalies in behavior, enabling our AI system to respond proactively and make timely interventions. Real-time monitoring and trigger identification allow for the detection of critical events, potential risks, or opportunities for immediate action.
Predictive Modeling & Personalized Strategies:
   Our Behavior Analytics algorithms incorporates predictive modeling techniques to forecast future behavior based on historical data and identified patterns. By leveraging machine learning algorithms, the system can predict individual behaviors. These predictions enable the AI system to develop personalized strategies, tailor its solutions, and optimize its decision-making processes to achieve desired outcomes.
Machine Learning & Reinforcement Learning:
   The AI system employs machine learning and reinforcement learning algorithms to continuously improve its analytical capabilities. By learning from around-the-clock inflow of data, the algorithms adapt and evolve, refining the AI model and personalized strategies over time. This iterative learning process enables the system to provide increasingly accurate predictions, personalized recommendations, and actionable insights, empowering the Xerbal model to make data-driven decisions.
   By utilizing a meticulously designed data model, the Xerbal behavior analytics AI approach revolutionizes behavior analysis. It leverages behavior patterns and correlations to enable real-time monitoring, trigger identification, predictive modeling, and personalized strategies specifically tailored to smoking cessation programs. Through the power of machine learning and reinforcement learning, this innovative approach effectively analyzes and predicts individual smoking behavior, identifies personalized intervention strategies, and unlocks valuable insights. By optimizing decision-making processes, Xerbal empowers individuals and healthcare professionals to achieve better outcomes in their efforts towards successful smoking cessation programs, ultimately improving public health and well-being.

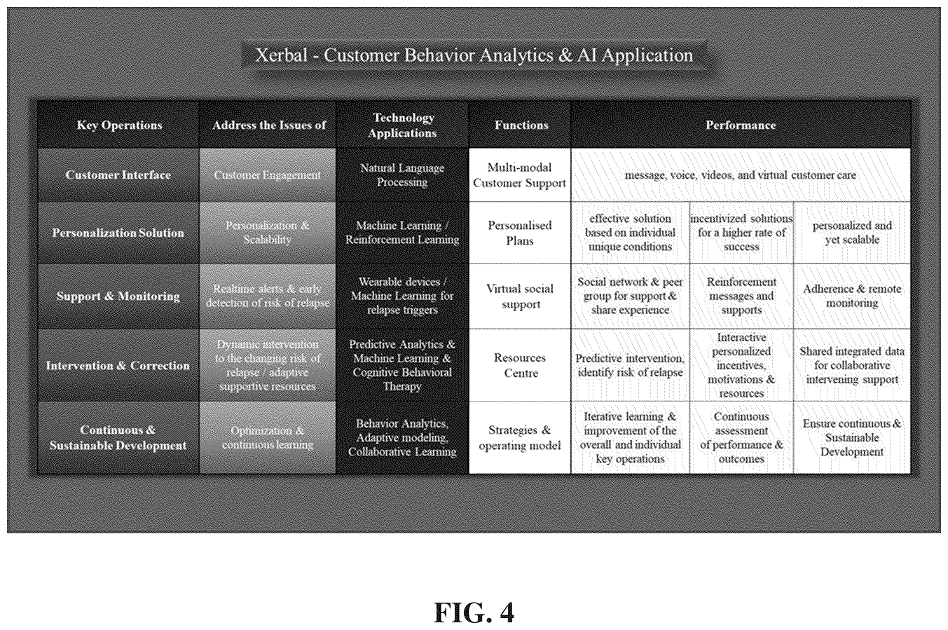

With respect to FIG. 4, FIG. 4 shows an overview of the Behavior Analytics & AI Applications in the Xerbal Model.

The Xerbal model may be implemented for either vape smokers or cigarette smokers.

Vape Smokers start with the highest concentration initially determined by questionnaires and gradually move towards lower concentrations of nicotine until the smoker's dependence on nicotine is eliminated.

Cigarette smokers will instill Nicotine Block solution into the filter. One drop blocks approximately 33%, 2 drops block approximately 66%, and 3 drops block approximately 99% of nicotine during inhaling.

In summary, it is a system and method for Smoking Cessation comprised of a 6-week process with user preferred natural products with a gradual nicotine reduction program. Plus, goal setting, lifestyle training & tailored counseling, it includes daily activities reminders driven by Xerbal AI program.

We discovered Mindset, smoking cessation education will lead to behavioral change, the key to smoking cessation success. Secondly the positive behavioral change needed to be supported & maintained by certain AI requirements with natural products.

Make lifestyle changes to reduce stress and improve quality of life, such as starting an exercise program or learning relaxation techniques. Vigorous exercise can enhance the ability to stop smoking and avoid relapse and helps to minimize or avoid weight gain.

Systems and methods are disclosed herein for classifying cigarette smoker's physical, mental, emotional, & psychological needs, such that specific products and services can effectively help smoking cessation, using a machine learning infusion algorithm.

The machine learning algorithm first uses the initial peer reviewed, clinically tested, scientifically validated questionnaire protocol.

Smokers' needs and records are classified, and high confidence classifications identified. All classifications metrics are submitted to the Product Regimen recommendation database for product and services selection. Requests are transmitted to analysts to generate training data that is added to the Smoking Cessation Product set. The process of classifying records and obtaining meta data validation thereof may then be perpetually repeated. This is further illustrated in the table below:

| | |
|---|---|
| Smoker's Key Metric | Scientific Validated standard Questionnaire |
| questionnaire #1 | forms: |
| | a) Quality of Life questionnaire |
| | b) Fagerstrom Tolerance Scale to |
| | determine smoking severity and |
| | nicotine dependence. |
| [symbol] | |
| Machine Learn Smoker's | Xerbal create a 3-tier support: |
| Behavior emotional | Body (Xerbal patented product) |
| | Mind (Personalized Xerbal Customer |
| | services) |
| | Soul (Sense of achievement as |
| | in Xerbal's mission) |
| & needs. #2 | With the Xerbal Personalized Plan, our |
| | customers will go through a gradual and |
| | managed process in adopting a new |
| | experience. That is out with the smoking |
| | addictions, and in with the new desired |
| | lifestyle. |
| [symbol] | |
| Assignment of Products | |
| and Services Programs | |
| [symbol] | |
| Smoker's Education, | |
| Service support | |
| [symbol] | |
| Algorithm Data | The advanced algorithms for behavioral |
| Refinement | analytics in smoking cessation aim to |
| | facilitate a transformative journey from the |
| | body to mind to soul. These algorithms |
| | analyze participant data, uncover behavioral |
| | patterns, and identify correlations to |
| | provide personalized recommendations. |
| | They employ predictive modeling |
| | techniques and real-time monitoring to |
| | support trigger identification, behavior |
| | replacement, emotional support, progress |
| | tracking, and personalized strategies. |
| | Through continuous learning |
| | facilitated by AI technology, the algorithms |
| | discover more personalized and effective |
| | strategies over time. The iterative data flow |
| | in the body-mind-soul behavioral analytics |
| | contributes to a comprehensive and holistic |
| | approach to smoking cessation. |
| [symbol] | |
| Back to #1 | |
| Smoker's Key Metric | |
| questionnaire #1 | |

Embodiments of the current system also address different needs and mindsets of different categories of individuals, including:
Consumers Age 18-30
   Curiosity-driven, open to trying new experiences, including different flavors.
   7.3% of female and 8.8% of male students currently smoke cigarettes.
   They also smoke shisha with a variety of flavors. 60.9% want to try and see what it's like and 39.1% smoke it because they like how it tastes.
   Education is needed to discourage inhaling petrochemicals.
Age 30-60
   Long-term heavy smokers who are motivated to quit and improve their health.
   Interested in starting a fitness routine and adopting a healthier lifestyle.
   Seeking alternatives to smoking due to the negative impact on their health and fitness goals.

Embodiments of the disclosed smoking cessation system include the use of e-cigarettes, or vapes, to wean a cigarette smoker off of full strength cigarettes and address their physical addiction. Vapes from Applicant include the following features and options:
25 flavors, multiple Nicotine levels 7% to 0%. The flavor system extracts from many tobacco species.
Experience all-natural certified ingredients.
Enjoy all-natural nicotine.
Embrace a clean approach, free from petrochemicals, artificial flavors, and sweeteners.
A smoother, more flavorful, and safer experience
GMP Compliance for Highest Quality
FDA registered manufacturing site
ISO 9001:2015 certified
Non-GMO and food-grade certification.
The following table provides further detail regarding the Applicant's vapes that are available for use in the smoking cessation system disclosed herein:

| | | Natural |
|---|---|---|
| Natural Flavors | Tobacco Types | Nicotine Percent |
| Fruit | Brightleaf tobacco | Country specific |
| Mint | (Virginia tobacco) | 7% to 0% |
| Candy | Broadleaf | |
| Menthol | Burley | |
| Tobacco flavored | Cavendish | |
| Wintergreen | Corojo | |
| Coffee | Criollo | |
| Spice | Dokha | |
| Combinations | Ecuadorian Sumatra | |
| of the above | Habano | |
| | Habano 2000 | |
| | Latakia | |
| | Maduro | |
| | Oriental Tobacco | |
| | Perique | |
| 36 Natural Flavor | Combination of Tobacco | 5 Nicotine levels |
| combinations | types Extracted into 5 | |
| developed. | different strengths: | |
| | Ultra-Light | |
| | Light | |
| | Medium (Regular) | |
| | Strong | |
| | Cigar like | |
| | 5 Taste/strengths levels | |
| 36 | 5 | 5 |
| | | 36X5X5 = 900 |
| | | Formulas in different |
| | | Flavor/Strengths |
| | | and Nicotine levels |

A second novel product of Applicant that is useful in the disclosed smoking cessation system is cigarette filter drops, which block nicotine and tar inhalation in filter cigarettes. Features and details include:
100% Food grade formula comprising Natural tobacco extract, Natural flavors, in a Syrup and Glycerin base.
One drop into the Cigarette filter blocks 33%, Two drops Blocks 66%, Three drops Blocks 99%.

Embodiments of the formulation and method of making the cigarette filter blocking drops are as follows:
providing an Aqueous base containing Corn syrup, water, preservative, glycerin with a pH adjustment of citric acid.

In a further aspect of the drops embodiment, there is provided a device for dropwise dispensing of a composition, the device comprising a container holding the composition as described herein.

It is one object of the present disclosure regarding the drops embodiment to provide optimum viscosity, namely between 3000 to 5000 centipoise (cps). These disclosed viscosity ranges provide a composition which has controllable drop size upon application.

Applicant has also determined that both the physical properties and rheology of the droplets must retain itself in the cigarette filter and not leak into the tobacco.

As such Applicants have found that one of the advantages of using Food grade Corn Syrup and Glycerin compositions is that it is possible to consistently control the droplet size & physical properties. The table below illustrates five different formulation embodiments for the drops:

| Ingredient | FN1 | FN2 | FN3 | FN4 | FN5 |
|---|---|---|---|---|---|
| Glycerol | 2.50% | 2.0% | 1.0% | 0.50% | 0% |
| Natural Flavor | 0.5% | 0.5% | 0.5% | 0.5% | 0.5% |
| Potassium sorbate | 0.25% | 0.25% | 0.25% | 0.25% | 0.25% |
| Sodium benzoate | 0.25% | 0.25% | 0.25% | 0.25% | 0.25% |
| Citric acid | 0.05% | 0.05% | 0.05% | 0.05% | 0.05% |
| Water | 9.45% | 9.45% | 9.45% | 9.45% | 9.45% |
| Corn syrup | 87% | 87.5% | 88.5% | 89% | 89.5% |

Natural Flavor is derived from Xerbal's proprietary Tobacco extraction method, which is disclosed and discussed further in pending U.S. patent application Ser. No. 18/423,185, entitled “Method for Extraction of Tobacco Flavors from Aged Tobacco Leaves,” a copy of which is incorporated herein by reference as if fully set forth herein. Natural Flavor may also be provided by the artful blending of other natural flavoring compounds.

Applicants note that Cellulose Acetate is soluble in many organic Solvents. Therefore, high purity ingredients are needed to avoid the presence of the following solvents: Acetone, Methyl-ethyl-ketone, Cyclohexanone, Diacetone alcohol, Methyl-formate, Methyl-acetate, Ethyl-acetate, Ethyl-lactate, Nitromethane Acetonitrile, N-Methylpyrrolidone, Dimethylformamide, Methyl glycol, Methyl-glycol-acetate, Tetrahydrofuran, Dioxane, Dioxolane, Methylene chloride Chloroform, Tetrachloroethane, Dimethyl-sulfoxide, and Propylene carbonate.

Embodiments disclosed herein also present Applicant's current and future development of an Artificial Intelligence supported smoking cessation system. The system works by leveraging the combined power of human bloggers and artificial intelligence technology. We will produce engaging and evidence-based content, sourced from the World's Top 50 organizations on health and wellness, to provide our audience with accurate information.

One embodiment will include an AI-Enhanced Online Quit Smoking Clinic with Human Doctor Collaboration, that that offers personalized guidance and support to help individuals quit smoking. In the initial stages, human doctors will collaborate with AI assistants to provide comprehensive care. As the AI system gains more professional data, experience, and stability, it will gradually assume a larger role in the process, thereby increasing efficiency and reducing the need for human intervention.

This growth of the AI system will enable Applicants to provide:
Tailor-Made Consultations
   The AI-powered virtual assistants will support human doctors in providing tailored consultations for customers based on their unique smoking habits, triggers, behaviors, and lifestyles.
Progress Tracking
   The AI-driven platform will monitor the user's progress and adapt the quit-smoking program as needed, ensuring that the approach remains effective and relevant to the individual's changing circumstances.
Online Support Groups
   The platform will offer AI-moderated forums and chat rooms to encourage user interaction, share experiences, and build a community that provides peer support and motivation.
Comprehensive & Personalized Content
   The AI system will curate and deliver customized educational materials, articles, and multimedia resources to help users better understand the quitting process and address their unique challenges.
Reminders and Notifications
   The AI technology will send timely reminders and motivational messages to users, keeping them engaged and focused on their goal of quitting smoking.

Embodiments disclosed herein are anticipated to recommend products and services for higher success rate and further prevention of relapse.

Search engines seek to identify documents that are relevant to the terms of a search query based on determinations of the subject matter of the identified documents. Another area in which smokers' classification is important is in product-related documents such as product flavors, product strengths, or other product-related natural content. The number of products available for sale constantly increases and the amount of data relating to a particular product is further augmented by social media posts.

Although some automatic classification methods are quite accurate, they are not progressive machine learning (i.e. AI) integrated. Often documents and Data identified or classified using automated methods are completely irrelevant. In addition, these methods are subject to manipulation by “spammers” who manipulate the word usage of content to obtain a desired classification but provide no useful content.

Of course, for a large volume of content, human classification of documents is not practical. The systems and methods described herein provide improved methods for incorporating both automated classification and human judgment in a highly effective manner.

The AI disclosed and/or contemplated herein features include the ability to:
Detect emotional state, smoker's state of mind, identify personalized coping strategies and relaxation techniques.
Analyze user behavior, taste preferences, and smoking frequency.
Create vape formulations based on user preferences and tailored to the smoking cessation needs of that user.
Analyze user interactions with the chatbot.
Develop algorithms for personalized content recommendations.
Create a content repository based on assessment scores and daily journals.
Design a notification system for reminders and updates.
Implement push notifications for important events.
Allow users to customize notification preferences.
Predictive analytics & Relapse risk identification
   Employ predictive analytics to forecast potential challenges or relapse risks.
   Identify high-risk periods and can offer proactive interventions.
Gamification Rewards
   Create an AI based gamification experience to incentivize quitting
   Determine rewards and badges and calculation to achieve them
UI/UX Enhancement, namely Screen and Experience Design
   Define the user journey for a smoother experience.
   Map out user flows for key features.
   Design theme
   Implement responsive design for various devices.
Cloud configuration, including AWS configuration.
   Authentication and authorization
   Real-time analytics configuration
   Wearable data configuration
   Data security and storage

The embodiments disclosed herein have been developed in response to the present state of the art AI capabilities.

Embodiments disclosed herein may be embodied as an apparatus, method, or computer program product. Accordingly, the disclosed embodiments may take the form of combining software and hardware aspects that may all generally be referred to herein as a “module” or “system.”

These computer program instructions may also be stored in a computer-readable medium that can direct a computer or other programmable data processing apparatus to function in a particular manner, such that the instructions stored in the computer-readable medium produce a recommendation instruction means which implement the function/act specified in the flowchart.

Embodiments can also be implemented in cloud computing environments. In this description and the following claims, “cloud computing” is defined as a model for enabling ubiquitous, convenient, on-demand network access to a shared pool of configurable computing resources (e.g., networks, servers, storage, applications, and services) that can be rapidly provisioned via virtualization and released with minimal management effort.

Processor(s) include one or more processors or controllers that execute instructions stored in memory device may also include various types of computer-readable media, such as cache memory.

Memory devices include various computer-readable media, such as volatile memory (e.g., random access memory may also include rewritable ROM, such as Flash memory.

Display device includes any type of device capable of displaying information to one or more users.

Interface(s) include various interfaces that allow computing devices to interact with other systems, devices, or computing environments.

In some embodiments, the modules and data of the system are implemented or accessed by the server system or some other entity that provides an interface to the server system.

The system includes smokers' training support data stored in a database. The training support data may include various data values used to train a classification model.

The training data is operational functions and data structures defining a machine learning algorithm and the state of a machine learning model. The machine learning algorithm used to implement may include any machine learning algorithm, including for example, a supervised or unsupervised learning algorithm, active learning algorithm, or the like.

In some embodiments, the high confidence classifications data are added to the training support data.

The unclear classifications, invalid classifications, will be eliminated or handled manually.

In some embodiments, and in particular with respect to early development of the database upon which the AI engine will operation, feedback received from a human analyst will be important. Our analyst is responsible for assessing data quality, overseeing data cleansing execution, developing classification rules, executing data classifications, performing validation checks, documenting the processes, and driving continuous improvement. With the expertise, our analyst contributes to ensuring the accuracy, consistency, and reliability of data, enabling the system to perform at its designed operations based on high-quality data.

In other embodiments, an analyst module, i.e., a computer program, may select classification values or categories of classification values on the basis on a percentage.

In certain data analysis scenarios, an analyst may need to select classification values or categories based on a percentage threshold. This approach allows for the identification and grouping of data into meaningful categories based on their relative proportions within the dataset. Here's an elaboration on how this process works:
1. Dataset Evaluation: The analyst begins by evaluating the dataset and understanding the nature of the data that needs to be classified. This may involve examining the data distribution, exploring different variables, and identifying any specific criteria or requirements for classification.
2. Percentage Threshold Determination: The analyst establishes a percentage threshold that will serve as the criterion for selecting classification values or categories. This threshold is typically determined based on the desired level of granularity or significance within the analysis. For example, the analyst may decide to select only those values or categories that account for at least 5% of the total dataset.
3. Calculation of Proportions: The analyst calculates the proportions of each value or category within the dataset. This can be done by dividing the count of occurrences for each value or category by the total count of observations in the dataset. The result is expressed as a percentage.
4. Selection Process: Using the determined percentage threshold, the analyst then selects the classification values or categories that meet or exceed the threshold. These values or categories are considered significant enough to be retained for further analysis, while those falling below the threshold may be grouped together or excluded from the analysis to maintain clarity and focus.
5. Grouping or Exclusion: Depending on the specific requirements of the analysis, the analyst may choose to group together the values or categories that fall below the percentage threshold. This consolidation helps to reduce complexity and improve interpretability. Alternatively, the analyst may exclude these values or categories altogether if they are deemed less relevant or contribute negligible insights to the analysis.
6. Iterative Process: The analyst may iterate through steps 3 to 5 above, adjusting the percentage threshold and reassessing the impact on the classification values or categories. This iterative process allows for fine-tuning the classification based on the desired level of granularity and significance.

By selecting classification values or categories based on a percentage threshold, analysts can effectively group and focus on the most relevant and significant data subsets. This approach aids in simplifying the analysis, highlighting important patterns or correlations, and facilitating decision-making based on the identified categories.

Further, actual records used to generate Meta data may be selected by analysts—either human or computer program—from a static pool of records.

As mentioned above, the machine learning algorithm may associate a confidence score with a Meta data output, a specified threshold may be added.

The AI system will store, retrieve, analyze, assimilate, recommend activities in a manner and fashion which the smoker will embrace. This will provide Smoking Cessation Clinics with a software application that will serve as the main intelligence tailored made for every individual resulting in higher success.

The design comprises:
a) Input algorithm contains numeric & text concepts.
b) Pre-programmed pre-stored Emotion Caring engine (Behavior modification recommendations.
c) The extract after machine learning allows expression of various patterns bypass human interventions.
d) This method comprises of a program that repeats itself in a single for-loop to receive information, calculate an optimal pathway from memory, and taking action; a 3-D storage area to store all data received.
e) This highly advanced AI program will have element objects go through a cascading (or recursive) loop. The cascading process will be comparing and analyzing for any patterns. If a pattern is found, then the AI program will generate a pattern object and store it in its respective pathway. This re-reinforces logical thoughts.
f) Real-time Monitoring: The AI system can continuously monitor the smoker's behavior, triggers, and progress in real-time. This can be achieved through integration with wearable devices, mobile applications, or other data sources. The system can collect data on smoking events, physiological indicators, location, and social interactions to provide a comprehensive understanding of the individual's smoking patterns.
g) Personalized Recommendations: Based on the collected data and analysis, the AI system can generate personalized recommendations for the smoker. These recommendations can include specific activities, coping mechanisms, stress reduction techniques, alternative behaviors, or support resources. The system can consider the individual's preferences, emotional state, and previous responses to tailor the recommendations for maximum acceptance and effectiveness.
h) Adaptive Learning: The AI system can continuously learn and adapt over time. It can incorporate feedback from the smoker regarding the effectiveness of the recommendations and use that information to refine its future suggestions. By leveraging machine learning techniques, the system can improve its accuracy and ability to predict and address individual needs.
i) Gamification and Rewards: To further motivate and engage smokers in their transformation journey, the AI system can incorporate gamification elements. It can introduce challenges, milestones, and rewards to encourage progress and celebrate achievements. The system can track and visualize the smoker's progress, providing a sense of accomplishment and fostering a positive mindset.
j) Integration with Support Systems: The AI system can integrate with existing smoking cessation clinics, healthcare providers, or support networks from the Xerbal Resources Center. It can facilitate communication and information sharing between the smoker, healthcare professionals, and support groups. This integration ensures a holistic approach to smoking cessation, combining the power of AI with human expertise and guidance.
k) Long-term Tracking and Analysis: The AI system can maintain a longitudinal view of the smoker's progress, tracking their behavior and outcomes over an extended period. This long-term tracking enables the system to identify trends, relapse patterns, and factors that contribute to successful cessation. The insights gained can inform continuous improvement in the system's recommendations and interventions.

Overall, the AI novelty by design for the smoking cessation system encompasses real-time monitoring, personalized recommendations, adaptive learning, gamification, integration with support systems, and long-term tracking and analysis. By incorporating these features, the system can provide tailored support to individuals, increase the chances of success in smoking cessation programs, and ultimately improve public health outcomes.

Additional anticipated advantages of the embodiments disclosed herein include that:
a) Billions of Permutations lengthy and time-consuming computation can be compressed by pre-programmed data memory.
b) Objects recognized by the AI program are called target objects and element objects are objects in memory that have strong association to the target object.
c) The AI program will collect all element objects from all target objects and determine which element objects to activate. This helped eliminate superfluous non-meaningful data. Thus, reduce computation time from days to minutes.
d) The embodiments disclosed herein can be used to write computer software without the need for human programmers. This means that human computer programmers are not needed to build computer software. This new form of writing computer software is that the codes are written by patterns from the Smokers environment. This will provide a truly clinical, data-supported approach to smoking cessation that goes beyond theoretical ideas.
e) Enhanced Efficiency through Pre-programmed Data Memory: The use of pre-programmed data memory enables the compression of lengthy and time-consuming computations involving billions of permutations. By storing relevant data in memory, the AI program can access and retrieve information more quickly, eliminating the need for repetitive calculations. This compression of computational tasks significantly reduces the time required for complex analyses, making the overall process more efficient and time-effective.
f) Target Objects and Element Objects for Data Filtering: The AI program distinguishes between target objects and element objects. Target objects are the specific entities or concepts of interest to the program, while element objects are objects in memory that exhibit a strong association with the target objects. By collecting and analyzing all element objects associated with the target objects, the AI program can determine which element objects are most relevant and activate them. This process helps filter out non-meaningful or superfluous data, streamlining the computation and analysis. As a result, computation time is further reduced from days to minutes, leading to faster and more efficient decision-making.
g) Automation of Computer Software Development: The present AI embodiments introduce the capability to write computer software without the need for human programmers. This means that the AI system can autonomously generate software code, eliminating the traditional reliance on human programmers for software development. The AI program leverages its learning capabilities and knowledge of patterns from the smoker's environment to generate code automatically. This breakthrough paves the way for more efficient and autonomous software development processes.
h) Clinical Approach to Software Development: The new form of writing computer software mentioned emphasizes a clinical approach that goes beyond theoretical ideas. Instead of relying solely on abstract concepts or hypothetical scenarios, the AI system generates code based on patterns derived from the smoker's environment. This clinical approach ensures that the software is grounded in practical observations and experiences, making it more effective in addressing the specific challenges of smoking cessation programs. By incorporating real-world patterns, the AI program can develop software solutions that are more closely aligned with the needs and behaviors of smokers.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments disclosed.

Insofar as the description above discloses any additional subject matter that is not within the scope of the claims below, the inventions are not dedicated to the public and the right to file one or more applications to claim such additional inventions is reserved.

We claim:

1. Nicotine-blocking drops for filter cigarettes comprising a composition of corn syrup, glycerin, water, and natural tobacco flavoring.

2. The nicotine-blocking drops composition of claim 1 further comprising potassium sorbate.

3. The nicotine-blocking drops composition of claim 1 further comprising sodium benzoate.

4. The nicotine-blocking drops composition of claim 1 further comprising citric acid.

5. The nicotine-blocking drops composition of claim 1 comprising:
at least 87% corn syrup by weight;
at least 9.45% water by weight; and
no more than 2.5% glycerin by weight.

6. The nicotine-blocking drops composition of claim 1 wherein the nicotine-blocking drops composition has a viscosity of between 3000 to 5000 centipoise (cps).

7. The nicotine-blocking drops composition of claim 1 wherein the composition does not contain any of the following solvents: Acetone, Methyl-ethyl-ketone, Cyclohexanone, Diacetone alcohol, Methyl-formate, Methyl-acetate, Ethyl-acetate, Ethyl-lactate, Nitromethane Acetonitrile, N-Methylpyrrolidone, Dimethylformamide, Methyl glycol, Methyl-glycol-acetate, Tetrahydrofuran, Dioxane, Dioxolane, Methylene chloride Chloroform, Tetrachloroethane, Dimethyl-sulfoxide, and Propylene carbonate.

8. The nicotine-blocking drops composition of claim 1 comprising:
about 87% corn syrup by weight;
about 9.45% water by weight;
about 2.5% glycerin by weight;
a preservative; and
citric acid.

9. The nicotine-blocking drops composition of claim 8 comprising about 0.5% natural tobacco flavoring by weight.

10. A method of reducing a cigarette smoker's nicotine inhalation comprising:
applying a nicotine-blocking composition to a cigarette filter;
wherein the nicotine-blocking composition comprises corn syrup, glycerin, water, and natural tobacco flavoring.

11. The method of claim 10 wherein the applying step comprises applying between one to three drops of the nicotine-blocking composition to the cigarette filter prior to smoking.

12. A method for smoking cessation regimen recommendation, the method comprising:
using a generative AI program to create a smoker classification model, which can further generate a personalized recommended product set to assist a smoker with smoking cessation;
training the generative AI program as to the smoker classification model regarding smoker behavior, outcomes, and effective smoking cessation products and strategies;
receiving, by the generative AI program, a validated portion of a training data set;
receiving, by the generative AI program, a smoker-record pairing, comprising data about an individual smoker's behavior, preferences, physical health, mental health, and spiritual health;
creating, by the generative AI program, a personalized recommended product set based upon the smoker-record pairing and the smoker classification model;
transmitting, by the generative AI program, the personalized recommended product set;
retraining, by the generative AI program, the smoker classification model using feedback data from a smoker who has received the personalized recommended product set.

13. The method of claim 12, further comprising:
identifying, by an analyst computer program, a product preference from the smoker-record pairing; and
retraining the generative AI program for the preference category using the product preference.

14. A system for classification, the system comprising one or more processors and one or more memory devices operably coupled to the one or more processors, the one or more memory devices storing executable and operational data effective to cause the one or more processors to:
train a classification model using a smoker database;
classify, using the classification model, a first record set to generate a first classification outcome product and service set;
receive a validated first record set and identify a category of product preferences from a set of classifier-record pairings;
retrain the classification model using the first record set and the set of classifier-record pairings; and
reclassify, using the classification model, a second record set to generate a second classification outcome set.

15. The system of claim 14, wherein the executable and operational data are further effective to cause the one or more processors to:
generate a prompt to generate a Meta data set.

16. The system of claim 14, wherein the executable and operational data are further effective to cause the one or more processors to:
generate a prompt to generate replacement records and updates.

17. The system of claim 16, wherein the executable and operational data are further effective to cause the one or more processors to:
add the validated portion to the Meta data set; and
access additional memory elements maintaining meta information used for updating.
